(19) United States
(12) Patent Application Publication
(10) Pub. No.: US 2002/0086281 A1
(43) Pub. Date: Jul. 4, 2002

(54) Detection of neurodegenerative diseases, in vitro screening assays

(76) Inventors: Timothy J. Greenamyre, Atlanta, GA (US); James J. Lah, Avondale Estates, GA (US); Julie Bennett-Desmelik, Tucker, GA (US); Allan I. Levey, Atlanta, GA (US)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 10/017,898

(22) Filed: Dec. 14, 2001

Related U.S. Application Data
(63) Continuation of application No. 09/247,537, filed on Feb. 9, 1999, now abandoned.
(60) Provisional application No. 60/074,729, filed on Feb. 13, 1998.

(51) Int. Cl. C12Q 1/00; C12N 5/08

(52) U.S. Cl. 435/4; 435/368

(57) ABSTRACT

Applicants have discovered that a variety of neurodegenerative diseases can be diagnosed, or screened for, by the analysis of a patient's mitochondrial metabolism, and thereby provide a screening assay for therapeutic drugs for the treatment of such neurodegenerative diseases.

### BACKGROUND OF THE INVENTION

Diagnosis of many neurodegenerative disorders is difficult and often based solely on clinical criteria. Even when specific genetic mutations are known to cause a disease, diagnostic genetic screening may not be feasible since a variety of different point mutations in a single gene may cause the disease. The assays of the current invention permit detection of the functional outcome of these mutations by measuring altered cellular physiology of blood samples taken from patients. Blood lymphocytes are transformed to lymphoblasts which are then compared to control lymphoblasts using microphysiometry. Once the lymphoblasts are available (a matter of days after receiving a blood sample), discriminative physiological testing can be performed within a few hours.

Development of therapeutics for neurodegenerative disorders is exceptionally difficult. For diseases caused by known genetic mutations, transgenic animal models are being developed. However, transgenic animal models are a slow and expensive process, and their relevance and utility is variable and often limited. Even when such models are developed successfully, they do not provide an efficient means of screening potential therapeutic agents. What is needed is an in vitro, biologically-relevant, rapid, high-throughput method for screening candidate therapeutics, both pharmacologic and genetic, in specific neurodegenerative disorders.

The primary technology applicants use to monitor cellular responses is a microphysiometer to measure a nonspecific endpoint and is broadly applicable to a variety of cellular functions. Applicants have discovered that a wide variety of different pathogenic genetic defects alter cellular metabolism in a manner that can be detected using microphysiometry.

Applicants have developed a method for rapid and efficient analysis of cellular defects caused by a variety of neurodegenerative diseases, including Alzheimer's disease (AD), Huntington's disease (HD), Parkinson's disease (PD) and mitochondrial disorders, such as Leber's hereditary optic neuropathy (LHON). Applicants' method employs a novel combination of cell culture and molecular biology techniques with rapid in vitro evaluation of cellular responses to therapeutic agents, both pharmacologic and genetic

### BRIEF DESCRIPTION OF THE INVENTION

Applicants have discovered that various neurodegenerative diseases show patterns of responses in proton excretion, when the appropriate cells are incubated in the presence of inhibitors of the mitochondrial electron transport chain or in the presence of calcium ionophores, and these patterns aid in the diagnosis of diseases including Alzheimer's disease, Huntington's disease, Leber's hereditary optic neuropathy, and Parkinson's disease. These patterns also provide assays for screening compounds suitable for the treatment of a neurodegenerative disease.

Abbreviations and Definitions

AD Alzheimer's disease

HD Huntington's disease

LHON Leber's hereditary optic neuropathy

PD Parkinson's disease

TTFA Thenoyltrifluoroacetone

MPTP 1-methyl-4-phenyl-1,2,3,6-tetrahydropyridine

MPP⁺ 1-methyl-4-phenylpyridinium

### BRIEF DESCRIPTION OF THE DRAWINGS

FIG. 1A: A graph showing results of microphysiometry of sporadic AD lymphoblasts, which shows apparent mitochondrial defects when cells are exposed to a complex I inhibitor, rotenone.

FIG. 1B: A graph showing results of microphysiometry of sporadic AD lymphoblasts, which shows apparent mitochondrial defects when cells are exposed to a complex III inhibitor, antimycin.

FIG. 1C: A graph showing results of microphysiometry of sporadic AD lymphoblasts, which shows a normal response to a complex II inhibitor, malonate.

FIG. 1D: A graph showing results of microphysiometry of sporadic AD lymphoblasts, which shows a normal response to a complex IV inhibitor, sodium azide.

FIG. 2A: A graph showing results of microphysiometry of PC12 cells transfected with two mutant forms of PS1, both known genetic causes of AD, which shows apparent mitochondrial defects when cells are exposed to a complex inhibitor, rotenone.

FIG. 2B: A graph showing results of microphysiometry of PC12 cells transfected with two mutant forms of PS1, both known genetic causes of AD, which shows a normal response to a complex II inhibitor, malonate.

FIG. 2C: A graph showing results of microphysiometry of PC12 cells transfected with two mutant forms of PS1, both known genetic causes of AD, which shows apparent mitochondrial defects when cells are exposed to a complex inhibitor, diuron.

FIG. 2D: A graph showing results of microphysiometry of PC12 cells transfected with two mutant forms of PS1, both known genetic causes of AD, which shows a reduced response to a complex IV inhibitor, sodium azide.

FIG. 3: A graph showing results of microphysiometry of HD fibroblasts, which shows altered calcium homeostasis after exposure to ionomycin. This altered response to ionomycin is due to mitochondrial dysfunction since it can be reproduced in control cells by rotenone.

FIG. 4A: A graph showing results of microphysiometry of LHON cells, which shows that the response to a complex I inhibitor, rotenone, is virtually absent in the 3460 mutation and markedly depressed in the 14459 mutation. Inhibitor was applied as indicated by the horizontal bar.

FIG. 4B: A graph showing results of microphysiometry of LHON cells, which shows a normal response to a complex II inhibitor, TTFA. Inhibitor was applied as indicated by the horizontal bar.

FIG. 4C: A graph showing results of microphysiometry of LHON cells, which shows that the response to a complex HI inhibitor, antimycin, is also markedly depressed. Inhibitor was applied as indicated by the horizontal bar.

FIG. 4D: A graph showing results of microphysiometry of LHON cells, which shows that the response to IV inhibitor, sodium azide, is mildly depressed in the 3460 cells and moderately depressed in the 14459 cells. Inhibitor was applied as indicated by the horizontal bar.

FIG. 4E: A graph showing results of microphysiometry of LHON cells, which shows that the response to ionomycin, a calcium ionophore, is also abnormal in both LHON cell lines. Inhibitor was applied as indicated by the horizontal bar.

FIG. 5A: A graph showing results of microphysiometry of PD lymphoblasts, which shows apparent mitochondrial defects when cells are exposed to a complex I inhibitor, rotenone.

FIG. 5B: A graph showing results of microphysiometry of PD lymphoblasts, which shows a normal response to a complex II inhibitor, malonate.

FIG. 5C: A graph showing results of microphysiometry of PD lymphoblasts, which shows a normal response to a complex III inhibitor, antimycin.

FIG. 5D: A graph showing results of microphysiometry of PD lymphoblasts, which shows a normal response to a complex IV inhibitor, sodium azide.

### DETAILED DESCRIPTION OF THE INVENTION

The present invention provides a method for aiding the diagnosis of, or screening for the risk of, a neurodegenerative disease in a subject, comprising the steps of

(a) providing a quantity of lymphoblasts from a subject to be evaluated;

(b) measuring acidification rate of the lymphoblasts, in the presence or absence of one or more compounds selected from the group consisting of an inhibitor of the mitochondrial electron transport chain and a calcium ionophore; and

(c) determining the presence of a pattern of responses in the stimulation of the acidification rate to a set of said compounds, that corresponds to a neurodegenerative disease.

In one embodiment of the present invention, the acidification rate is measured with a microphysiometer.

In another embodiment of the present invention, the neurodegenerative disease is Alzheimer's disease having the pattern of responses comprising a defective response in the stimulation of the acidification rate in the presence of complex I inhibitor, a normal response in the acidification rate in the presence of complex II inhibitor, a defective response in the stimulation of the acidification rate in the presence of complex III inhibitor, and a normal response in the acidification rate in the presence of complex IV inhibitor.

In another embodiment of the present invention, the neurodegenerative disease is Huntington's disease having the pattern of response comprising a defective response in acidification rate in the presence of a calcium ionophore.

In another embodiment of the present invention, the neurodegenerative disease is Leber's Hereditary Optic Neuropathy (LHON) having the pattern of responses comprising a defective response in the acidification rate in the presence of complex I inhibitor, a normal response in the acidification rate in the presence of complex II inhibitor, a defective response in the acidification rate in the presence of complex III inhibitor, a defective response in the acidification rate in the presence of complex IV inhibitor, and a defective response in the acidification rate in the presence of a calcium ionophore.

In another embodiment of the present invention, the neurodegenerative disease is Parkinson's disease having the pattern of responses comprising a defective response in the acidification rate in the presence of complex I inhibitor, a normal response in the presence of complex II inhibitor, a normal response in the acidification rate in the presence of complex III inhibitor, and a normal response in the acidification rate in the presence of complex IV inhibitor.

In another embodiment of the present invention, a compound suitable for the treatment of a neurodegenerative disease is isolated or identified by the methods of the present invention, wherein the compound substantially reverses the defective effect of one or more inhibitors of the mitochondrial electron transport chain on the acidification rate in patient cells.

In another embodiment of the present invention, a compound suitable for the treatment of a neurodegenerative disease is isolated or identified by the methods of the present invention, wherein the compound substantially reverses the defective effect of a calcium ionophore on the acidification rate in patient cells.

The rate at which cells excrete protons (the acidification rate) is closely linked to the rate at which they convert nutrients to energy. Because energy metabolism is coupled to ATP consumption, any event that perturbs ATP levels, such as receptor activation or calcium influx, causes a change in energy metabolism and, therefore, an alteration in proton excretion. In addition, receptor activation may also directly affect proton excretion by altering Na/H exchange. A typical microphysiometer, such as the Cytosensor™ (Molecular Devices), uses a silicon-based sensor to measure the rate of proton excretion from 10⁴-10⁶ cells. Cells are contained in a 3 μL flow chamber in aqueous contact with the pH-sensitive surface of a light-sensitive potentiometric sensor. Flow of medium (with low buffering capacity) and test compounds are determined by a series of microprocessor-controlled pumps. The extracellular acidification rate is determined by measuring the decrease in pH that occurs as acidic metabolites accumulate during brief halts in the flow of medium. The degree of stimulation of the basal acidification is calculated and plotted as a per cent of the basal acidification rate. The sensor takes a voltage measurement that is linearly related to pH. Typically, during a 30-60 sec halt in flow, pH will decrease less than 0.1 pH unit. Reproducible and robust responses are obtained to metabolic inhibitors, growth factors and ionophores in lymphoblast lines. Because virtually any manipulation alters proton excretion, it is relatively easy to determine the types of stimuli to which disease cell lines respond abnormally.

A single microphysiometer can contain up to 8 cell chambers, thereby allowing great flexibility in experimental design. For example, a 4-point dose-response curve can be generated simultaneously in control and disease cell lines. Alternatively, 8 drugs can be screened simultaneously in a disease cell line. Because the microphysiometric responses are obtained in essentially real time, this technique is suitable for relatively high-throughput screening of targeted pharmacological agents.

Alzheimer's Disease (AD)

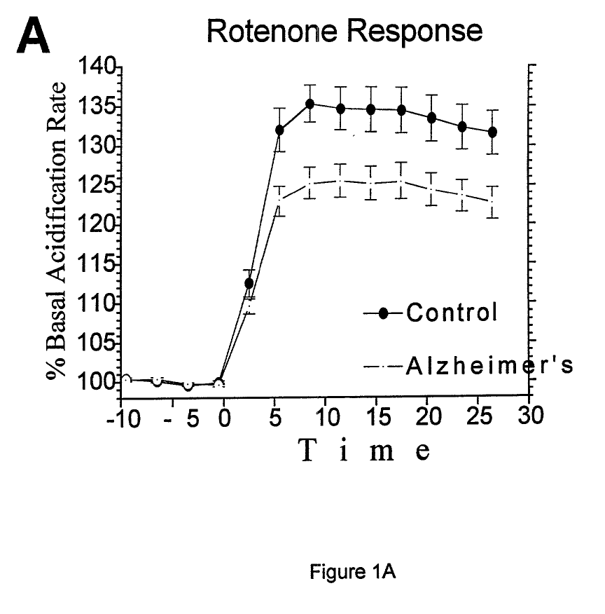

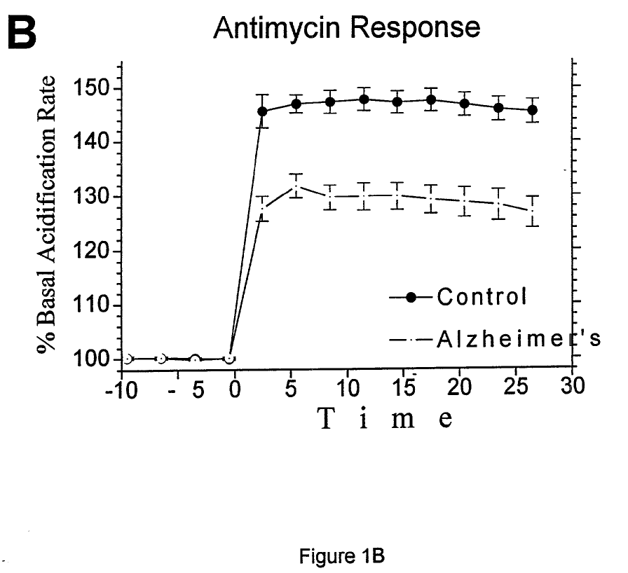

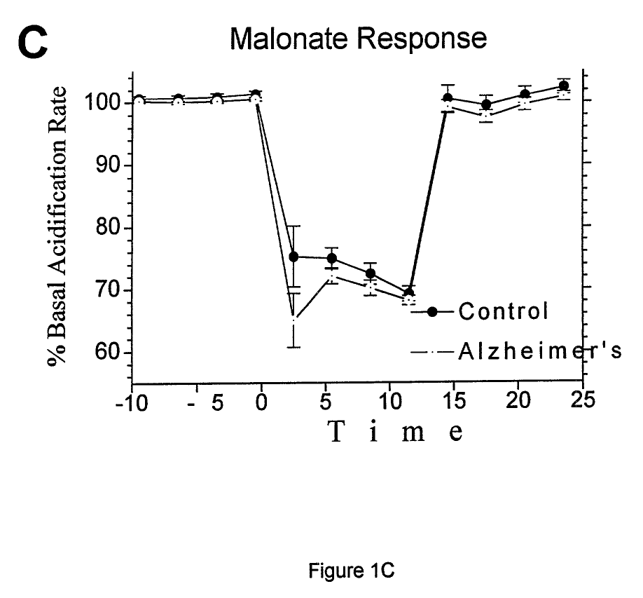

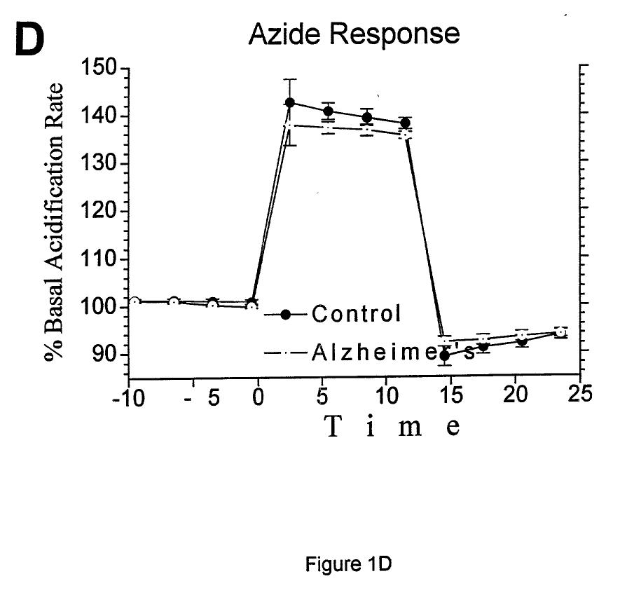

Approximately 5-10% of AD cases are caused by autosomal genetic mutations, i.e., familial AD, and the remaining 90% of cases are of undefined cause and are termed “sporadic AD.” Sporadic AD lymphoblasts have markedly blunted microphysiometric responses to the mitochondrial complex I inhibitor, rotenone (FIG. 1A), and to the complex III inhibitor, antimycin-A (FIG. 1B). Responses to the complex II inhibitor, malonate (FIG. 1C), and to the complex IV inhibitor, sodium azide (FIG. 1D) do not differ substantially from controls. These results indicate that there is chronic impairment of mitochondrial function in AD and they provide a simple diagnostic test for sporadic AD.

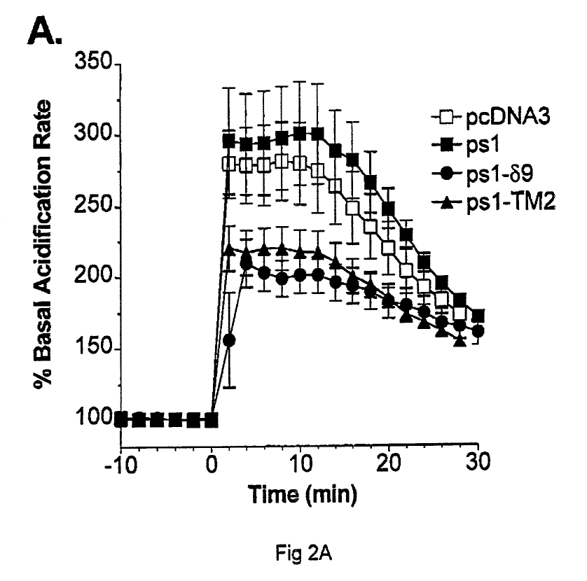

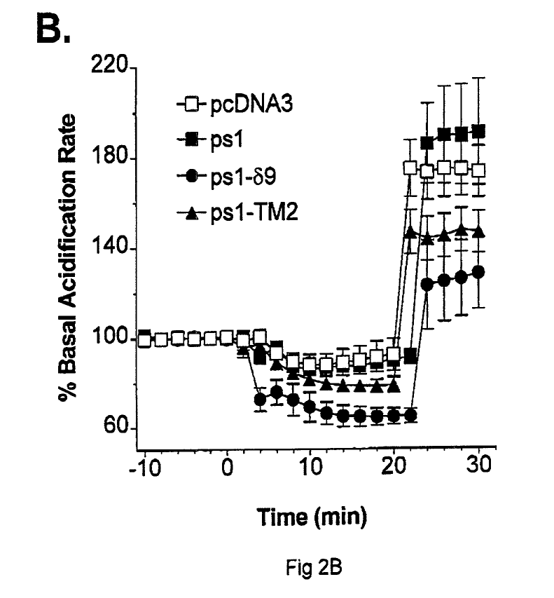

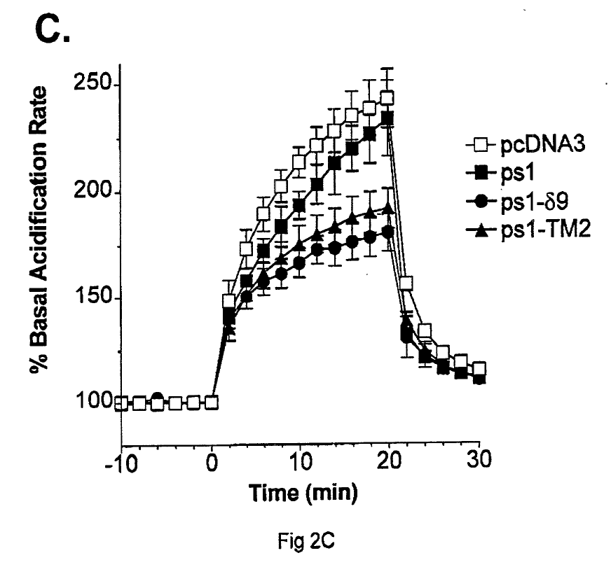

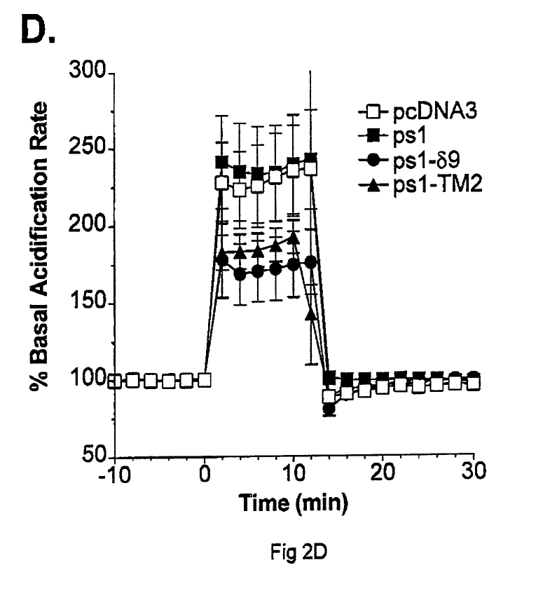

In PC12 cells transfected with the most common genetic mutations that cause familial AD with onset at early age (presenilin-1), a similarly depressed response to rotenone (FIG. 2A) and to the complex III inhibitor diuron (FIG. 2C) were found. These results indicate that microphysiometry is suitable for the diagnosis of genetic forms of AD.

In addition, these results show that microphysiometry provides a method for screening potential therapeutic strategies, both pharmacologic and genetic. The ability of new drugs or genetic therapies to normalize the AD responses predicts therapeutic efficacy.

Huntington's Disease (HD)

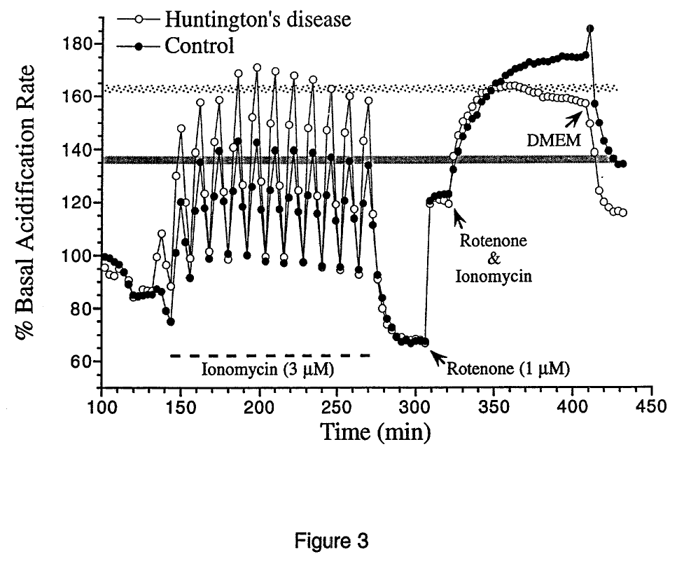

A leading hypothesis of Huntington's disease pathogenesis suggests that the causative mutation in the protein, huntingtin, leads secondarily to mitochondrial dysfunction and altered calcium homeostasis. HD cells have abnormal responses to the calcium ionophore, ionomycin (FIG. 3). This altered response to ionomycin is due to mitochondrial dysfunction since it can be reproduced in control cells by rotenone (FIG. 3). These results show that microphysiometry provides a method for screening potential therapeutic strategies, both pharmacologic and genetic. The ability of new drugs or genetic therapies to normalize the HD responses predicts therapeutic efficacy.

Leber's Hereditary Optic Neuropathy (LHON)

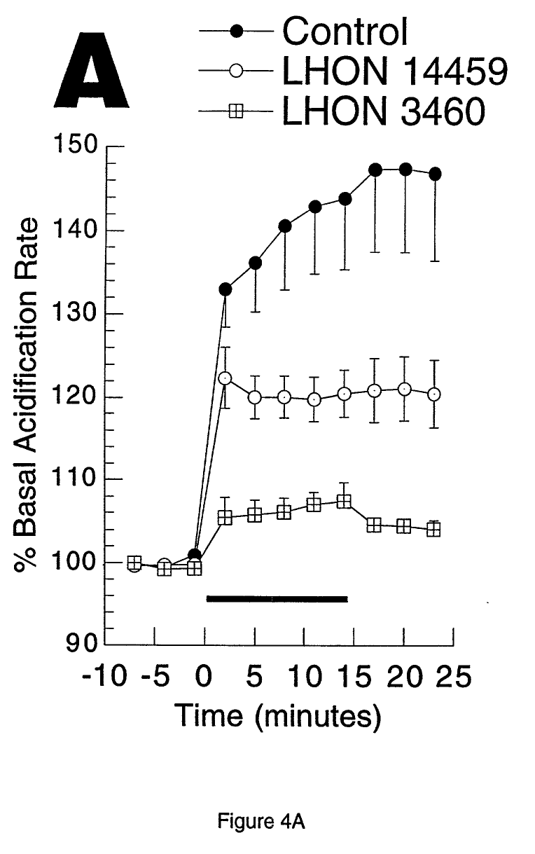

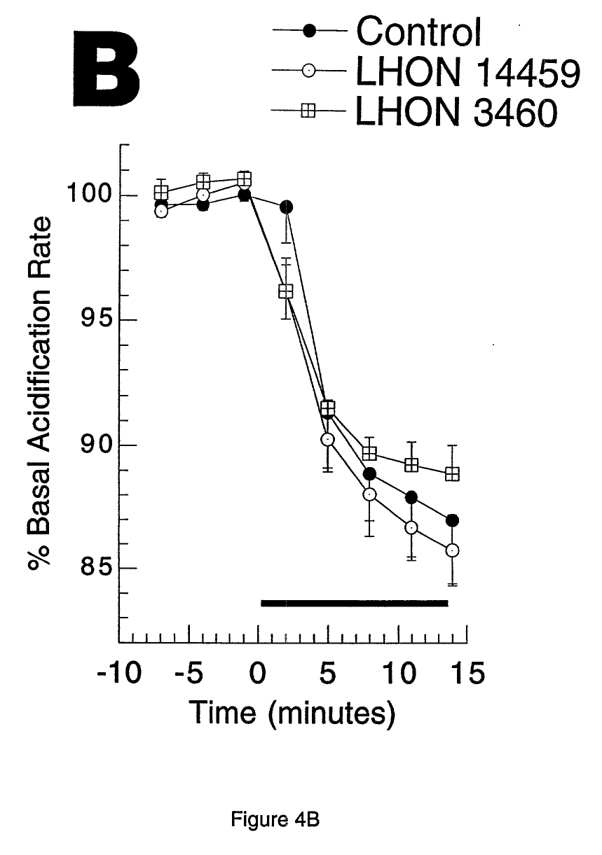

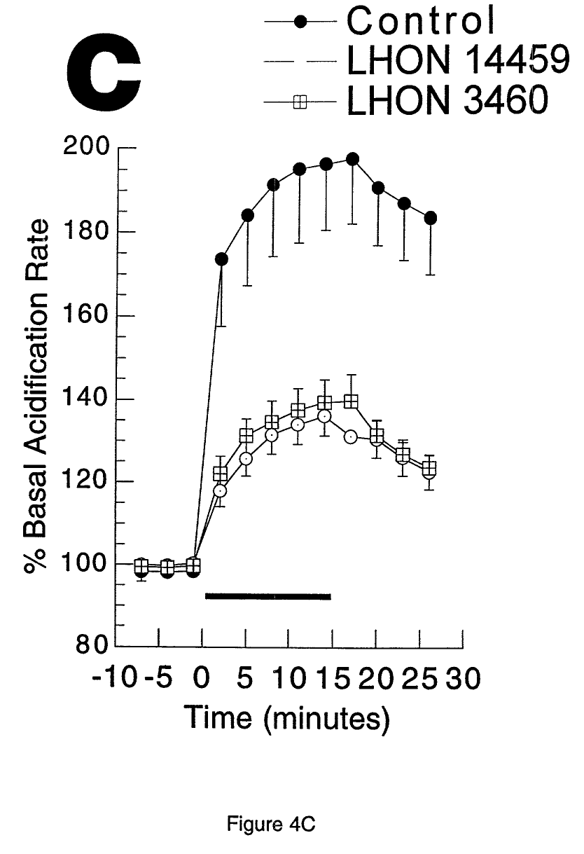

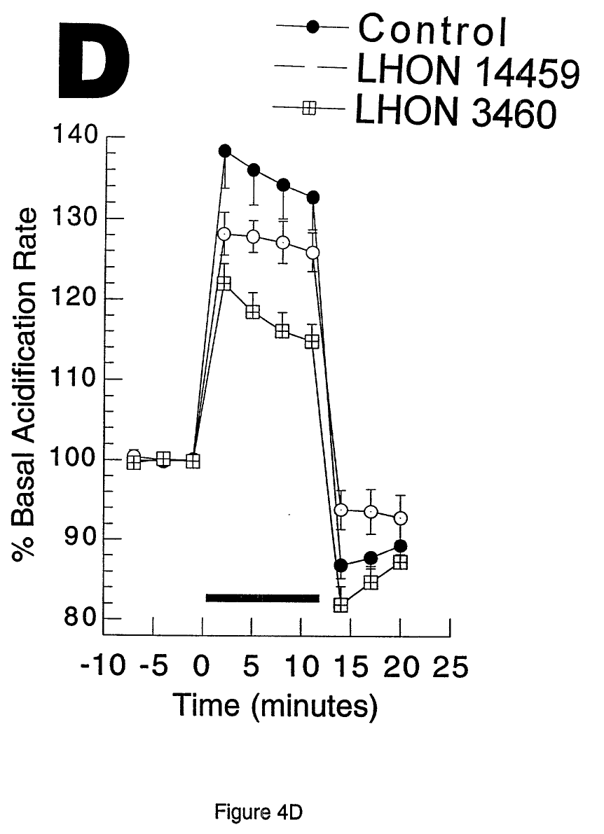

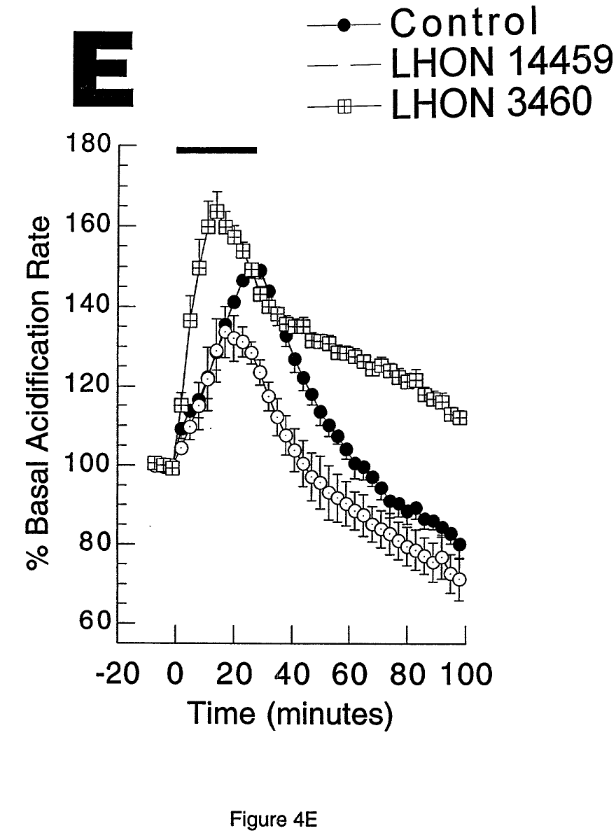

Leber's hereditary optic neuropathy (LHON) is caused by mutations in mitochondrial genes that encode subunits of complex I of the mitochondrial electron transport chain. Two of these mutations are the 3460 and 14459 mutations, both of which are detected by microphysiometry. The microphysiometric response to a complex I inhibitor, rotenone, is virtually absent in the 3460 mutation and markedly depressed in the 14459 mutation (FIG. 4A). In contrast, there is no difference between control and patient lymphoblast responses to a complex II inhibitor, TTFA (FIG. 4B). Responses to a complex III inhibitor, antimycin, are also markedly depressed (FIG. 4C). Responses to a complex IV inhibitor, sodium azide, are mildly depressed in the 3460 cells and moderately depressed in the 14459 cells (FIG. 4D). Responses to ionomycin, a calcium ionophore, are also abnormal in both LHON cell lines (FIG. 4E).

These results provide an alternative, non-genetic method for diagnosis of suspected LHON. These results also show that microphysiometry provides a method for screening potential therapeutic strategies, both pharmacologic and genetic. The ability of new drugs or genetic therapies to normalize the LHON responses predicts therapeutic efficacy.

Parkinson's Disease (PD)

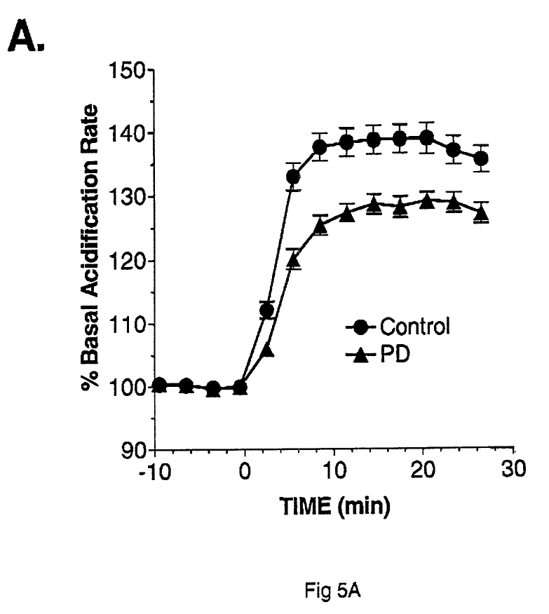

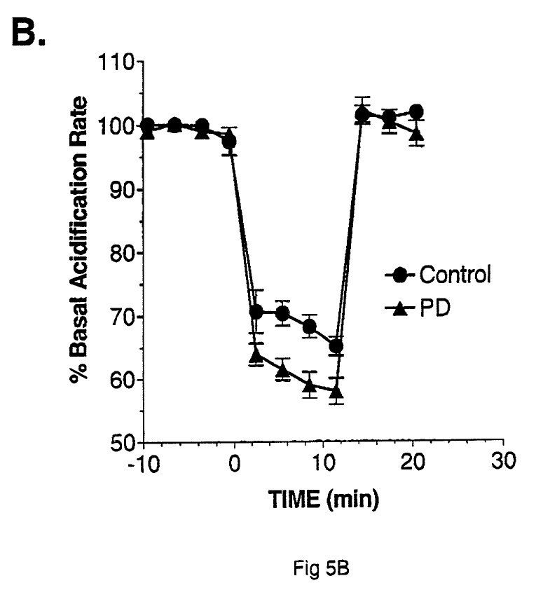

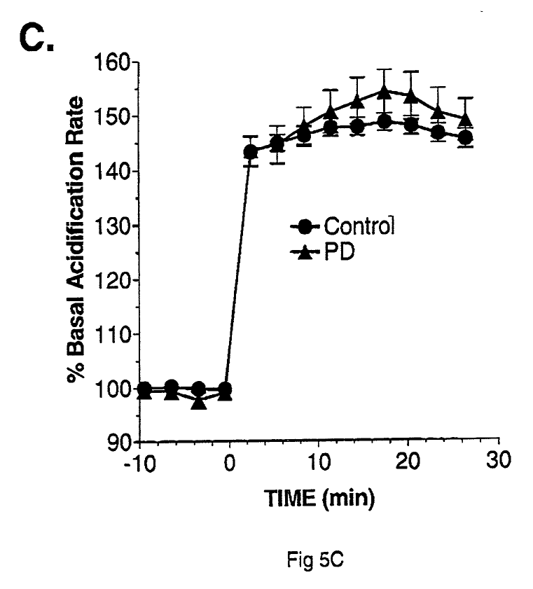

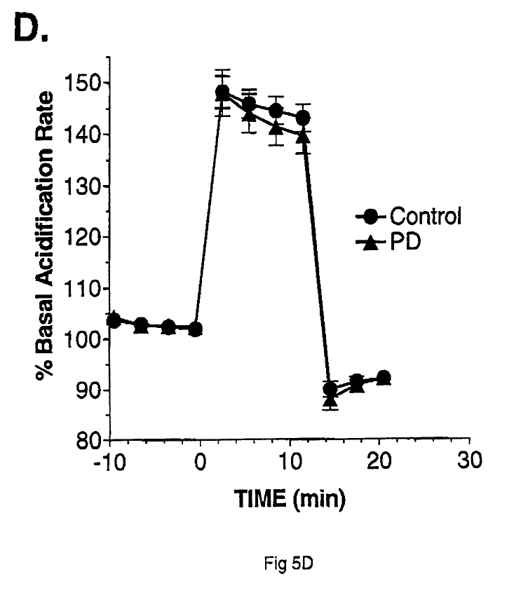

Currently, the cause of PD is unknown, although one leading hypothesis proposes that altered mitochondrial complex I activity may be responsible. PD lymphoblasts have markedly blunted microphysiometric responses to the mitochondrial complex I inhibitor rotenone I (FIG. 5A). Responses to the complex II inhibitor malonate (FIG. 5B), to the complex III inhibitor antimycin (FIG. 5C), and to the complex IV inhibitor sodium azide (FIG. 5D) do not differ substantially from controls. These results indicate that there is chronic impairment of mitochondial function in PD and they provide a simple diagnostic test for PD. These results also show that microphysiometry provides a method for screening potential therapeutic strategies, both pharmacologic and genetic. The ability of new drugs or genetic therapies to normalize the PD responses predicts therapeutic efficacy.

Metabolic Inhibitors Suitable for the In vitro Assay of Mitochondrial Defects

There are various inhibitors of the electron transport chain in mitochondria, and they serve as useful probes of the biochemical mechanisms underlying the metabolism of mitochondria. Inhibitors of the electron transport chain inhibit the release of protons to the outside of the mitochondrial cell membrane. Applicants have discovered that each neurodegenerative disease has a distinctive response to one or more inhibitors of the electron transport chain. Applicants have selected a limited number of inhibitors, but it will be understood that many other inhibitors of mitochondrial metabolism are suitable for the in vitro assay of mitochondrial defects. Suitable inhibitors include, but are not limited to, inhibitors of each of the complexes of the electron transport chain, e.g., complexes I, II, III or IV. Other compounds that disrupt the release of protons to the outside the mitochondrial cell membrane are also suitable for the in vitro assay of mitochondrial defects, e.g., calcium ionophores that disrupt cytosolic calcium levels.

Suitable inhibitors of complex I of the electron transport chain (ETC) include, but are not limited to, rotenone, 1-methyl-4-phenyl-1,2,3,6-tetrahydropyridine (MPTP) or 1-methyl-4-phenylpyridinium (MPP⁺). One preferred complex I inhibitor is rotenone. A suitable range of concentrations of rotenone for the in vitro assay ranges from about 1.0 nM to about 10 μM, preferably about 100 nM. A suitable range of concentrations of MPTP for the in vitro assay ranges from about 100 nM to about 1 mM. A suitable range of concentrations of MPP⁺ for the in vitro assay ranges from about 100 nM to about 1 mM, preferably about 10 μM.

Suitable inhibitors of complex II of the electron transport chain include, but are not limited to, thenoyltrifluoroacetone (TTFA), malonate or 3-nitropropionic acid. One preferred complex II inhibitor is malonate. A suitable range of concentrations of TTFA for the in vitro assay varies from about 1 μM to about 100 mM. A suitable range of concentrations of malonate for the in vitro assay varies from about 100 μM to about 100 nM, preferably about 30 mM. A suitable range of concentrations of 3-nitroproprionic acid for the in vitro assay varies from about 100 μM to about 100 mM.

Suitable inhibitors of complex III of the electron transport chain include, but are not limited to, antimycin-A or myxothiazol. One preferred complex III inhibitor is antimycin-A, also known as antimycin. A suitable range of concentrations of antimycin for the in vitro assay varies from about 1 μg/ml to about 1 mg/ml, preferably about 100 μg/ml. A suitable range of concentrations of myxothiazol for the in vitro assay varies from about 10 ng/ml to about 1 mg/ml.

Suitable inhibitors of complex IV of the electron transport chain include, but are not limited to, sodium azide or cyanide. One preferred inhibitor of complex IV is sodium azide. A suitable range of concentrations of sodium azide for the in vitro assay varies from about 100 nM to about 100 mM, preferably about 3 mM. A suitable range of concentrations of cyanide for the in vitro assay varies from about 100 mM to about 100 mM.

Oxamic acid is preferably used to block lactate dehydrogenase. A suitable range of concentrations of oxamic acid for the in vitro assay varies from about 100 μM to about 100 mM, preferably about 5 mM.

Suitable calcium ionophores for disrupting cytosolic calcium levels include, but are not limited to, ionomycin or calcimycin (A23187). One preferred calcium ionophore is ionomycin. A suitable range of concentrations of ionomycin for the in vitro assay varies from about 10 nM to about 100 μM, preferably about 3 μM. A suitable range of concentrations of A23187 varies from about 10 nM to about 100 μM.

Patient Cell Lines

Controls: Control cell lines were generated from blood samples obtained from individuals without neurologic or mitochondrial disorders.

LHON: Previous work at Emory University has identified causative mitochondrial DNA mutations in LHON patients. Well-characterized LHON patient cell lines have been generated and banked. These cell lines have been characterized by molecular genetic, cellular respiration and enzymology studies. The patient cell lines were obtained from the Emory University Clinical Research Center (Emory University, Atlanta, Ga.).

Alzheimer's disease: Lymphoblast cell lines were generated from blood samples obtained from patients followed in the Emory Alzheimer's Disease Center. All patients have been evaluated by detailed neurological and neuropsychometric examinations. All individuals given a diagnosis of AD met the National Institutes of Neurological Disorders and Stroke-Alzheimer's Disease and Related Disorders Association (NTINDS-ADRDA) criteria.

Huntington's disease: Cell lines (both lymphoblasts and fibroblasts) were generated from blood samples obtained from patients followed in the Emory Huntington's Disease Clinic. All patients carry a clinical diagnosis of HD based on family history, clinical symptoms and detailed neurological evaluation; diagnosis was confirmed by genetic testing.

Parkinson's disease: Cell lines were generated from blood samples obtained from patients followed in both the Emory Movement Disorders Clinic and the Alzheimer's Disease Center. The diagnosis of PD was made by Movement Disorders specialists based on clinical presentation, detailed neurologic assessment and response to anti-parkinsonian medications.

Transformation of Blood Lymphocytes to Lymphoblasts as Cell Lines for In Vitro Assay of Mitochondrial Defects

Patient lymphocytes are immortalized using the technique of Neitzel H, Human Genetics 73:320-326 (1986). In brief, the lymphocytes are removed from the buffy coat layer following Histopaque density separation. The lymphocytes are then incubated with Epstein-Barr virus (B95-8, ATCC Access Number CRL 1612) in transforming media consisting of RPMI-1640, 20% heat-inactivated fetal bovine serum (FBS), 2 μg/ml cyclosporin, 2 mM L-glutamine, and penicillinistreptomycin. Following transformation, the lymphoblasts are maintained in RPMI-1640 media supplemented with 15% heat-inactivated FBS and 110 μg/ml sodium pyruvate.

Generation of Transfected PC12 Cells as Models of AD

Rat adrenal pheochromocytoma PC 12 cells were stably transfected with PS1 cDNA cloned behind the CMV promoter in pcDNA3. See, e.g., Lah, J. J. et al., J. Neurosci. 17:1971-1980 (1997), herein incorporated by reference. Briefly, the cells were plated onto 10 cm dishes at a density of 50,000/cm² and grown in a 37° C. incubator with 5% carbon dioxide. After 24 hrs, 20 μg of plasmid DNA was added to each plate in a 1 ml solution of 25 mM BES, 140 mM sodium chloride, 0.750 mM sodium phosphate, 125 mM calcium chloride, pH 6.95. The cells were then returned to a 37° C. incubator with 3% carbon dioxide for an additional 18-24 hrs. The media was then replaced with fresh growth media and returned to 5% carbon dioxide. Stable PC12 transfectants were selected with 400 μg geneticin and single colonies were isolated. Clonal cell lines were established by limiting dilution cloning to ensure growth from an individual cell, and PSI protein expression confirmed by western blotting and immunocytochemistry.

In Vitro Assay of Mitochondrial Defects with Microphysiometry

Lymphoblasts are suspended in agarose entrapment solution at a density of 5×10⁵ cells/10 μl, then are plated onto chamber inserts, and allowed to solidify for 20 minutes. Cells are then placed into the microphysiometer recording chambers containing a low-buffering-capacity recording buffer made of high glucose DMEM, pH 7.40, with 44.4 mM sodium chloride replacing sodium bicarbonate in the media. In all cases, except where stated, sodium pyruvate is present in the recording buffer at 110 μg/ml. Recording buffer flows across the cells at a rate of 100 μl/min. The pump cycles on for 2 min 30 sec, followed by a 30 sec off period. The rate of acidification is calculated as the slope of a linear least squares fit to the relation of pH versus time during the pump's off period. Cells are equilibrated in the running media for 50-120 min prior to drug exposure. Cells are run in duplicate for each treatment. The pH of all final drug solutions was checked to ensure that the drug did not alter the pH of the running buffer.

While the foregoing specification teaches the principles of the present invention, with examples provided for the purpose of illustration, it will be understood that the practice of the invention encompasses all of the usual variations, adaptations, and modifications, as come within the scope of the following claims and its equivalents.

What is claimed is:

1. A method for aiding the diagnosis of, or screening for the risk of, a neurodegenerative disease in a subject, comprising the steps of
(a) providing a quantity of lymphoblasts from a subject to be evaluated;
(b) measuring acidification rate of the lymphoblasts, in the presence or absence of one or more compounds selected from the group consisting of an inhibitor of the mitochondrial electron transport chain and a calcium ionophore; and
(c) determining the presence of a pattern of responses in the stimulation of the acidification rate to a set of said compounds, that corresponds to a neurodegenerative disease.

2. The method of claim 1, wherein the acidification rate is measured with a microphysiometer.

3. The methods of claim 1 or 2, wherein the neurodegenerative disease is Alzheimer's disease having the pattern of responses comprising a defective response in the stimulation of the acidification rate in the presence of complex I inhibitor, a normal response in the acidification rate in the presence of complex II inhibitor, a defective response in the stimulation of the acidification rate in the presence of complex III inhibitor, and a normal response in the acidification rate in the presence of complex IV inhibitor.

4. The methods of claims 1 or 2, wherein the neurodegenerative disease is Huntington's disease having the pattern of response comprising a defective response in acidification rate in the presence of a calcium ionophore.

5. The methods of claims 1 or 2, wherein the neurodegenerative disease is Leber's Hereditary Optic Neuropathy (LHON) having the pattern of responses comprising a defective response in acidification rate in the presence of complex I inhibitor, a normal response in acidification rate in the presence of complex II inhibitor, a defective response in the acidification rate in the presence of complex III inhibitor, a defective response in the acidification rate in the presence of complex IV inhibitor, and a defective response in the acidification rate in the presence of a calcium ionophore.

6. The methods of claims 1 or 2, wherein the neurodegenerative disease is Parkinson's disease having the pattern of responses comprising a defective response in acidification rate in the presence of complex I inhibitor.

7. A compound suitable for the treatment of a neurodegenerative disease, wherein the compound substantially reverses the defective effect of one or more inhibitors of the mitochondrial electron transport chain on the acidification rate, as measured by the method of claim 1.

8. A compound suitable for the treatment of a neurodegenerative disease, wherein the compound substantially reverses the defective effect of a calcium ionophore on the acidification rate, as measured by the method of claim 1.
